(19) United States
(12) Patent Application Publication
(10) Pub. No.: US 2003/0144719 A1
(43) Pub. Date: Jul. 31, 2003

(54) Method and apparatus for shielding wire for MRI resistant electrode systems

(76) Inventor: Volkert A. Zeijlemaker, Landgraaf (NL)

Correspondence Address: MEDTRONIC, INC., 710 MEDTRONIC PARKWAY NE, MS-LC340, MINNEAPOLIS, MN, 55432-5604, US

(21) Appl. No.: 10/059,594

(22) Filed: Jan. 29, 2002

(51) Int. Cl. A61N 1/05

(52) U.S. Cl. 607/122

(57) ABSTRACT

The present invention discloses apparatus and methods for use with implantable medical system. In one embodiment a medical electrical lead is disclosed comprising an electrode wire and a shield wire adjacent to at least a portion of the electrode wire, the electrical lead is capable of insertion within a body and the shield wire is electrically separated from the electrode wire. In another embodiment of the invention a method of shielding a medical electrical lead is disclosed. The method comprises receiving electromagnetic energy within a shield wire that is adjacent at least a portion of the lead. The energy is dissipated into a living body, thus reducing the amount of electromagnetic energy received by the medical electrical lead.

### FIELD OF THE INVENTION

This invention relates generally to a method and apparatus for electrically stimulating a heart, and, more particularly, to a method and apparatus for reducing the effects of an electromagnetic field on the operation and safety of implantable medical devices.

### DESCRIPTION OF THE RELATED ART

Since their earliest inception some forty years ago, there has been a significant advancement in body-implantable electronic medical devices. Today, these implantable devices include therapeutic and diagnostic devices, such as pacemakers, cardioverters, defibrillators, neural stimulators, drug administering devices, among others for alleviating the adverse effects of various health ailments. Today's implantable medical devices are also vastly more sophisticated and complex than their predecessors, and are therefore capable of performing considerably more complex tasks for reducing the effects of these health ailments.

A variety of different implantable medical devices (IMD) are available for therapeutic stimulation of the heart and are well known in the art. For example, implantable cardioverter-defibrillators (ICDs) are used to treat patients suffering from ventricular fibrillation, a chaotic heart rhythm that can quickly result in death if not corrected. In operation, the ICD continuously monitors the electrical activity of a patient's heart, detects ventricular fibrillation, and in response to that detection, delivers appropriate shocks to restore normal heart rhythm. Similarly, an automatic implantable defibrillator (AID) is available for therapeutic stimulation of the heart. In operation, an AID device detects ventricular fibrillation and delivers a non-synchronous high-voltage pulse to the heart through widely spaced electrodes located outside of the heart, thus mimicking transthoracic defibrillation. Yet another example of a prior art cardioverter includes the pacemaker/cardioverter/defibrillator (PCD) disclosed, for example, in U.S. Pat. No. 4,375,817 to Engle, et al. This device detects the onset of tachyarrhythmia and includes means to monitor or detect progression of the tachyarrhythmia so that progressively greater energy levels may be applied to the heart to interrupt a ventricular tachycardia or fibrillation. Numerous other, similar implantable medical devices are available, including programmable programmable pacemakers, nerve stimulation systems, physiological monitoring devices, and the like.

Modern electrical therapeutic and/or diagnostic devices for the heart and other areas of the body generally include an electrical connection between the device and the body. This connection is usually provided by a medical electrical lead. Such a lead normally takes the form of a long, generally straight, flexible, insulated conductor. At its proximal end, the lead is typically connected to a connector of the electrical therapeutic and diagnostic device, which may be implanted within the patient's body. Generally an electrode is located at or near the distal end of the lead and is attached to, or otherwise comes in contact with, the body. In the case of an implantable pacing or defibrillation system, leads are electrically coupled to tissue within the heart or within a coronary vessel.

In the case of cardiac applications, a tip electrode is may be anchored to the heart tissue by means of a screw-in lead tip that can be inserted into the heart tissue. Another fixation mechanism utilizes tines that are affixed to the trebeculae of the heart. This provides a physical connection of the lead to the heart tissue. Alternatively, when an electrode is positioned within a vessel, the electrode can be shaped so that it may be wedged between the walls of the vessel. Other anchoring means are known in the art. In any of these embodiments, the area of tissue making contact with the electrode is relatively small.

Problems may be associated with implanted leads when a patient comes in contact with alternating electromagnetic fields. Such fields can induce an electric current within a conductor of the lead. In fact, in the presence of electromagnetic fields, an implanted electrical lead acts as an antenna, resulting in an electrical current that flows from the lead, through body tissue, and back to the IMD. If this current is large, damage to the device can occur. Additionally, the implanted medical device can sense the imposed voltage on the lead and react inappropriately, resulting in the wrong therapy being administered. Examples of inappropriate therapy modification include changing the rate or thresholds associated with pacing pulses.

Alternating electrode fields are often used in medical diagnosis techniques such as Magnetic Resonance Imaging (MRI), which is a technique for producing images of soft tissue within the human body. MRI scanners from many different sources are now well known and commercially available. Magnetic resonance spectroscopic imaging (MRSI) systems are also known and are herein intended to be included within the terminology “MRI” system or scanner. These techniques can give valuable diagnostic information without the need for invasive surgery, but also subject the patient to significant alternating electromagnetic fields, resulting in the risks described above.

For the foregoing reasons, various mechanisms have been developed in the prior art to protect the IMD circuitry when in the presence of a magnetic field. For example, U.S. Pat. No. 5,217,010 describes a device for monitoring or pacing a patient that operates within a MRI system. The device uses an RF filtering and shielding to attenuate voltages on the leads resulting from the high frequency RF signals produced in the MRI, and further to protect circuitry within the device.

As noted above, the large currents induced by electromagnetic fields can result in damage to, or improper operation within, device circuitry. These induced currents can also result in injury to the patient. Because the tissue area associated with electrode contact may be very small, the current densities may be high, resulting in heating that can injure the tissue. Moreover, a sudden burst of radio-frequency energy can cause an electric pulse within the lead that could send the heart into fibrillation. For both of these reasons, this type of high-density current may be fatal.

There is therefore a need for improved methods and apparatus that reduce the detrimental effects that are possible when a patient with an implantable electrical lead is subjected to an electromagnetic field.

### SUMMARY OF THE INVENTION

In one embodiment of the present invention a medical electrical lead is disclosed comprising an electrode conductor and a shield conductor adjacent to at least a portion of the electrode conductor, wherein the electrical lead is capable of insertion within a body and the shield conductor is electrically separated from the electrode conductor, the shield conductor is adapted to at least partially electrically shield the electrode conductor.

In another embodiment of the invention an apparatus is disclosed comprising an implantable medical device and an electrical lead connected to the implantable medical device. A shield wire is disposed within the lead, the shield wire adapted to at least partially electrically shield the electrical lead.

In another embodiment of the invention a method of shielding a medical electrical lead is disclosed. The method comprises receiving electromagnetic energy within a shield wire that is adjacent at least a portion of the lead. The energy is dissipated into a patients body, to at least partially reduce an amount of electromagnetic energy received by the medical electrical lead.

Yet another aspect of the present invention is a method of manufacturing a shielded medical lead. The method comprises providing a lead having at least one electrode wire and at least one shield wire, the at least one shield wire being adapted to receive electromagnetic waves. A layer of insulative material is applied over the at least one electrode wire and the at least one shield wire, wherein the shield wire is not in direct electrical communication with the electrode wire.

### BRIEF DESCRIPTION OF THE DRAWINGS

The invention may be understood by reference to the following description taken in conjunction with the accompanying drawings, in which like reference numerals identify like elements, and in which:

FIG. 1 schematically illustrates an implanted medical device within a human body;

FIG. 2 schematically illustrates an embodiment of an implanted medical device with an endovenous epicardial lead positioned adjacent the left ventricle of a heart;

FIG. 3 schematically illustrates an embodiment of an implantable medical device with a tip electrode located at the distal end of the lead;

FIG. 4 is a table showing various named regions of the electromagnetic spectrum along with their respective wavelength, frequency and energy ranges;

FIG. 5 is a generalized drawing of an embodiment of the present invention;

FIG. 6 is a generalized drawing of an embodiment of the present invention;

FIG. 7 schematically illustrates an embodiment of an implantable medical device comprising features of the present invention;

FIGS. 8A-8C schematically illustrate cross sectional views of embodiments of a lead comprising features of the present invention;

FIG. 9 schematically illustrates a cross sectional view of an embodiment of an implantable medical device comprising features of the present invention;

FIG. 10 shows a cross-sectional view of a portion of the embodiment of the invention shown in FIG. 9; and

FIG. 11 is an electrical model of the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but, on the contrary, the intention is to cover all modifications, equivalents, and alternatives falling within the scope of the invention as defined by the appended claims.

### DETAILED DESCRIPTION OF SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

Embodiments of the present invention provide for means to reduce the potentially harmful effects that can occur when a medical implantable device is subjected to electromagnetic energy fields.

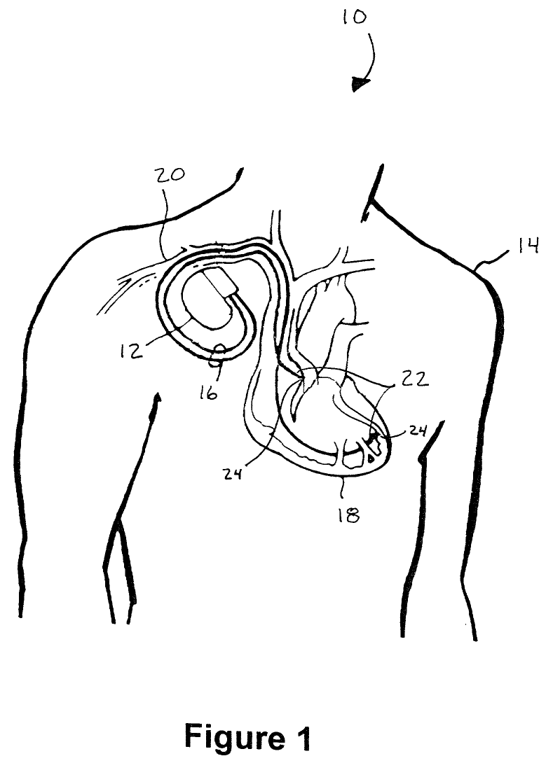

FIG. 1 illustrates an implantable medical device (IMD) system 10, which includes an implantable electronic device 12, such as a pacemaker, defibrillator, or the like, that has been implanted in a patient's body 14. One or more pacemaker leads, collectively identified with reference numeral 16, are electrically coupled to the pacemaker 12 in a conventional manner and extend into the patient's heart 18 via a vein 20.

Located generally near the distal end 22 of the leads 16 are one or more exposed conductive electrodes 24 that are attached to the heart 18 tissue, sensing cardiac activity, delivering electrical pacing stimuli to the heart 18, or providing a stimulating voltage to defibrillate the heart 18. The contact area between the electrodes 24 and the heart 18 tissue is very small as compared, for example, to the contact area between the device 12 and the body 14.

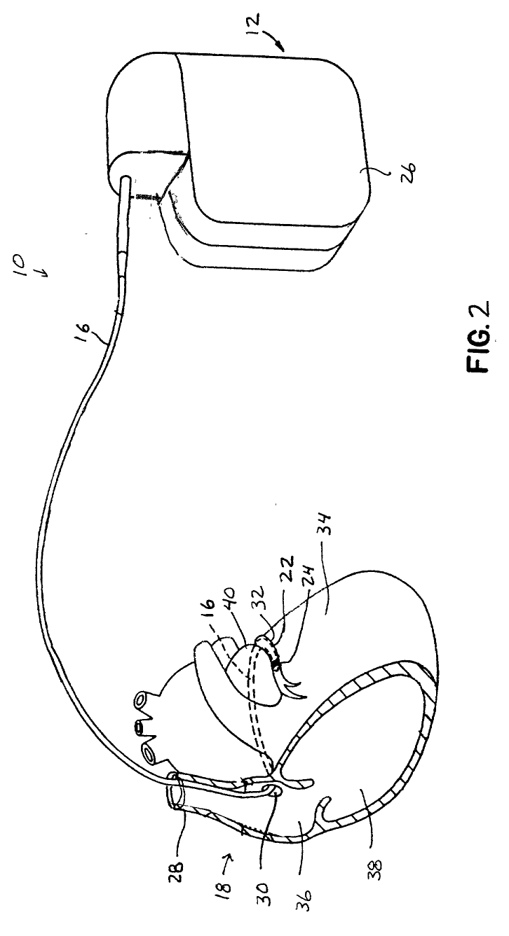

FIG. 2 illustrates an implantable medical device (IMD) system 10, which includes an implantable electronic device 12, such as a pacemaker or defibrillator. The device 12 is housed within a hermetically sealed, biologically inert outer canister or housing 26, which may itself be conductive. One or more leads 16 are electrically coupled to the device 12 and extend to a point adjacent the patient's heart 18 via a vein, typically the superior vena cava vein 28. This embodiment illustrates a lead 16 proceeding from the right atrium 36, through the coronary sinus 30 and its distal end 22 positioned within a cardiac vein 32 that is adjacent to the left ventricle 34 of the heart 18. An electrode 24, for example, a ring electrode, is placed in contact with the cardiac vein 32 to provide the necessary electrical proximity between the device 12 and the heart 18. Leads can also be placed within the right atrium 36 and the right ventricle 38 of the heart 18. This illustration shows the relatively small contact area between the electrode 24 and the tissue of the vein 32.

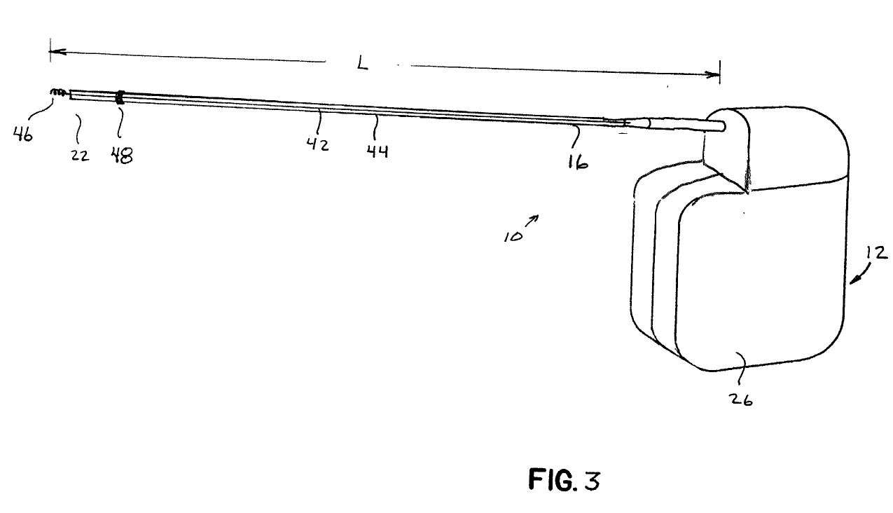

FIG. 3 schematically illustrates an embodiment of an implantable medical system 10 having an electrical lead 16 extended to its length L. The electrical lead 16 is shown as a cross section to show the electrode wire 42 surrounded by an insulator 44. A tip electrode 46 is shown at its distal end 22, and a ring electrode 48 is shown located on the surface of the lead 16. One embodiment of a tip electrode 46 comprises a helical coil that can be affixed directly to heart tissue and is particularly useful in attaching the lead 16 within an atrium or ventricle chamber wall of a heart 18, such as shown in FIG. 1. The ring electrode 48 can comprise a section of the lead 16 outer surface and can comprise a protrusion, such as a ridge having a larger diameter than the remainder of the lead 16. Ring electrodes 48 are particularly useful when the lead 16 is placed within a vein, such as the cardiac vein 32 shown in FIG. 2. The lead 16 can comprise more than one electrode wire 42, and in an embodiment such as shown in FIG. 3, separate electrode wires 42 can be connected to the tip electrode 46 and the ring electrode 48, respectively.

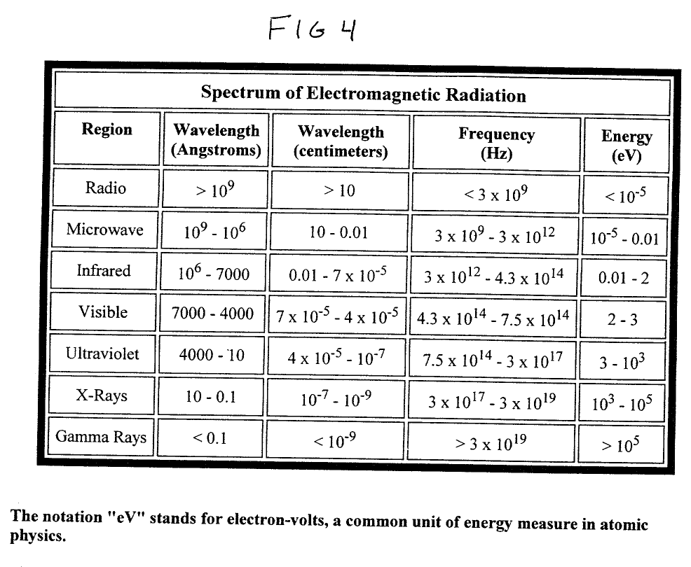

FIG. 4 is a table showing various named regions of the electromagnetic spectrum along with their respective wavelength, frequency and energy ranges. As the frequency increases, the amount of energy that is transmitted increases as well. The magnet in an MRI system is rated using a unit of measure known as a “Tesla.” Another unit of measure commonly used with magnets is the “gauss”, where 1 Tesla equals 10,000 gauss. The magnets in use today in MRI systems are in the 0.5 Tesla to 2.0 Tesla range, or 5,000 to 20,000 gauss. Although magnetic fields greater than 3 Tesla have not currently been approved for use in medical imaging, much more powerful magnets, up to 60 Tesla, are in use in research. An MRI system of 1 Tesla will operate at a frequency of approximately 42 MHz or 42×10E6 hertz. This is within the radio region of the electromagnetic radiation spectrum and is commonly referred to as radio-frequency (RF) energy. In the search for better diagnostic capabilities, MRI systems utilizing stronger magnets capable of generating increasing amounts of RF energy are being developed. The greater the level of RF energy transmitted, the greater the risk of damaging a patient by inducing electrical currents within an implanted lead.

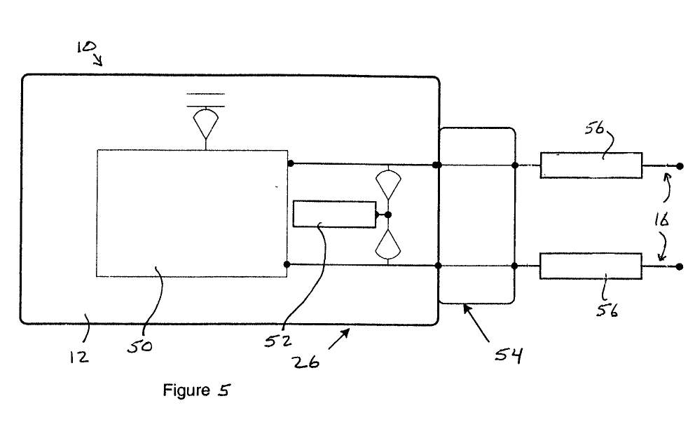

FIG. 5 is a generalized drawing of an embodiment of the present invention comprising an implantable system 10 having an electronic device 12 enclosed within a housing 26. Electronics 50 and a power supply 52 are contained within the device 12 and are connected to a pair of electrical leads 16 by a connector 54. Two separate leads 16 coupled to a connector 54 (also shown in FIG. 6) are referred to as unipolar leads. Passive or active components 56 may be incorporated within the leads 16 to decrease the effects of the magnetic field on the lead, as disclosed in commonly-assigned U.S. patent application entitled “Apparatus and Method for Shunting Induced Current in an Electrical Lead”, Docket Number P-9698, filed on Oct. 31, 2001, and a continuation-in-part of that application having the same title and filed on even date herewith, both incorporated herein by reference in its entirety.

Embodiments of the present invention provide for shielding a wire associated with the leads 16, such that the quantity and/or amplitude of the currents induced by electromagnetic signals are reduced or dissipated.

FIG. 6 is a generalized drawing of an embodiment of the present invention comprising an implantable system 10 having an electronic device 12 enclosed within a housing 26. Electronics 50 and a power supply 52 are contained within the device 12 and are connected to a pair of electrical conductors 58 that are joined within a single lead 16 by a connector 54. The embodiment of a single lead 16 as shown in FIG. 6 can be referred to as a bipolar lead, embodiments may alternatively comprise multiple bipolar leads. Passive or active components 56 are incorporated within the electrical conductors 58 of the lead 16 to alter the effects of electromagnetic fields on the system 10. Embodiments of the present invention provide for shielding a wire associated with the leads 16, such that the quantity and/or amplitude of the currents induced by electromagnetic signals are reduced or dissipated.

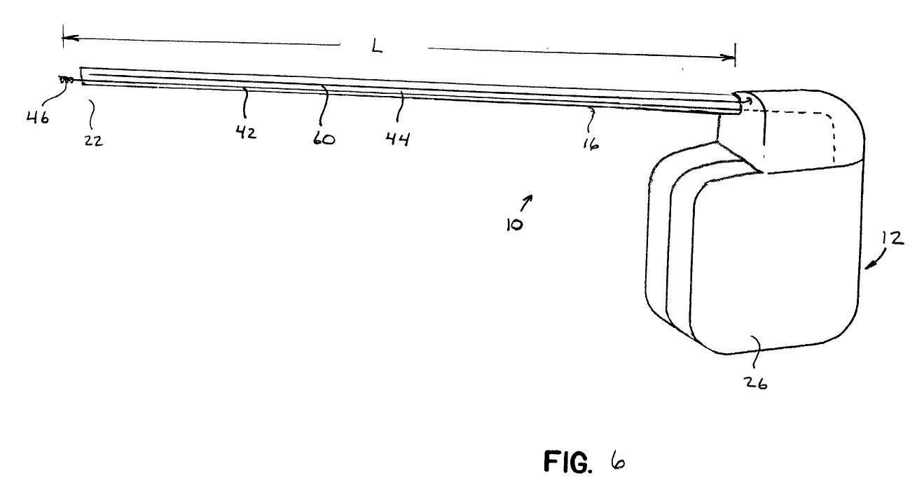

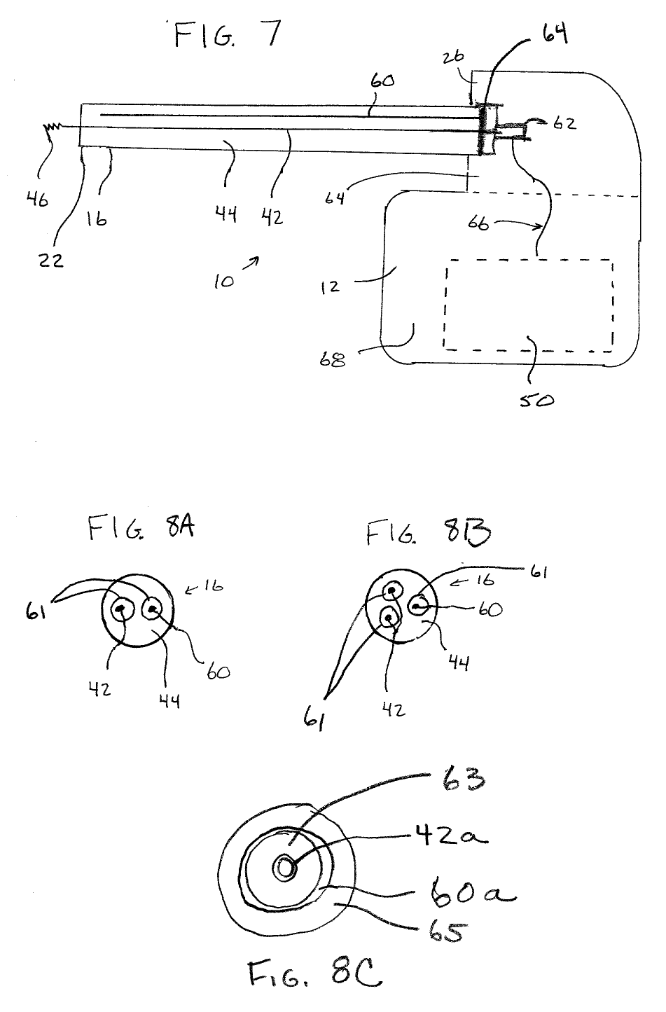

FIG. 7 schematically illustrates an embodiment of an implantable medical system 10 comprising features related to embodiments of the present invention. The electrical lead 16 is shown as a cross section to show the electrode wire 42 surrounded by an insulator 44. A tip electrode 46 is shown at the distal end 22 of the electrode wire 42. The particular embodiment shown comprises a tip electrode 46 comprising a helical coil that can be affixed directly to heart tissue and is particularly useful in attaching the lead 16 within an atrium or ventricle chamber wall of a heart 18, such as shown in FIG. 1. Other types of electrodes can be used including alternate tip electrode designs and ring electrodes. A shield wire 60 is shown within the interior of the lead 16, insulated from direct electrical contact with the electrode wire 42 and with the electrode 46. The shield wire 60 may be in contact with the medical device 12 housing 26, which may act as surface area for dissipation of energy (e.g., due to electromagnetic signals) received by the shield wire 60. In some embodiments portions of the shield wire 60 may be in direct contact with a patient's body, such as an area where the shield wire 60 contacts the housing 26. In embodiments where the shield wire 60 can contact the patient's body, the shield wire 60 may comprise a material, for example, titanium, that is compatible with the body tissues and fluids, sometimes referred to as a biocompatible material. Shield wire 60 may be twisted or coiled around the other wires present, such as the electrode wire 42. In other embodiments the shield wire 60 may partially or totally surround the active wires such as the electrode wire 42.

The electrode wire 42 is typically connected to the electronics 50 within the device 12 by means of a connection ring 62 or another connector located within a connection block 64. A connection wire 66 provides an electrical path from the lead 16 to the electronics section 68 of the device 12. In one embodiment, the shield wire 60 may generally not be in direct contact with the electrode wire 42 or the electrode 46, and therefore is not in direct electrical communication with the various electrical parts of the system 10.

As discussed above, when a conductor such as electrode wire 42 is placed within a magnetic field, a current in induced within the conductor which flows into tissue via tip electrode 46. However, according to the current invention, shield wire 60 receives some of the radio-frequency energy otherwise received by electrode wire 42. The shield wire 60 acts as an antenna for receiving radio-frequency energy and dissipates the energy in a manner designed to be safe for the patient. In one embodiment, shield wire 60 may be coupled to one or more conductive surface areas of implantable device housing 26 or other ring electrodes as discussed further below so that energy may be safely dissipated. This minimizes the risk of heart tissue damage due to heating of the localized contact area between the electrode 46 and the heart.

An implantable medical lead 16 must be flexible to be inserted within the body of a patient. The shield wire 60 may typically comprise a material and be of a size that will not substantially alter the stiffness of the lead 16. The term “not substantially alter” within one embodiment of the present application means that a lead with a shield wire retains enough flexibility to enable the lead to be implanted within the body of a patient. The shield wire 60 may have a twisted or coiled configuration within the lead 16, thus enabling an increased range of movement that the lead 16 can withstand.

FIGS. 8A and 8B are cross-sectional views of electrical leads of various embodiments of the invention. An electrode wire 42 and shield wire 60 run parallel to each other, separated from each other by an insulating material 44, thereby insulating the electrode wire 42 from the shield wire 60. FIG. 8A illustrates a unipolar lead 16 having a single electrode wire 42 and a shield wire 60 surrounded by an insulating material 44. FIG. 8B illustrates a bipolar lead 16 having two electrode wires 42 and a shield wire 60 that are surrounded by an insulating material 44. The insulating material restricts electrical communication between the electrode wires 42 and the shield wire 60. The shield wire 60 is typically comprised of the same material and general size as the electrode wires 42, so that it will not significantly restrict the flexibility or alter the diameter of the lead 16, although other materials and sizes may also be used.

FIG. 8C is a cross-sectional view of yet another embodiment of the inventive lead. In this embodiment, electrode wire 42a is a coiled conductor. Shield wire 60a is also a coiled conductor co-axially positioned with respect to electrode wire 42a. A tube 63 formed of an electrically insulating material such as silcone or polyurethane surrounds electrode wire 42a, and electrically isolates that wire from shield wire 60a. Insulated lead body 65 surrounds the shield wire and is formed of a biocompatible insulating material. In a similar multi-polar lead embodiment, additional conductors provided to couple to additional electrodes may be co-axially positioned within the lead body.

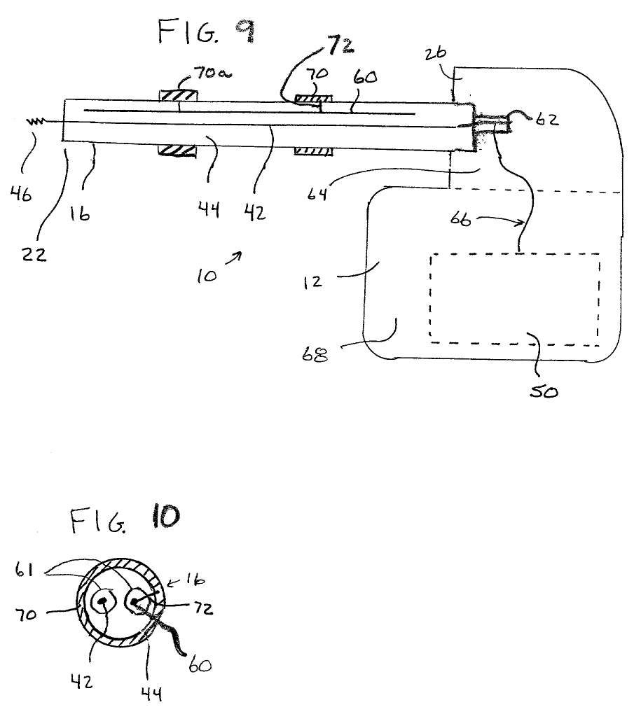

FIG. 9 schematically illustrates a cross sectional view of an alternate embodiment of the present invention comprising an implantable medical system 10 having an electrical lead 16. The electrical lead 16 comprises an electrode wire 42 surrounded by an insulator 44 and a tip electrode 46 is shown at the distal end 22 of the lead 16. A shield wire 60 located within the lead 16 comprises an electrically conductive material and is in electrical contact with at least one electrically conductive surface positioned adjacent an external surface of the lead body, such as a ring electrode 70 located on an exterior area of the lead 16. In one embodiment there may be multiple ring electrodes located along the length of the lead 16 to ground the shield wire 60 to the patient's body. The shield wire 60 may be twisted or coiled around the other wires present, such as the electrode wire 42.

The shield wire 60 acts as an antenna for radio-frequency waves. The shield wire 60 may collect and dissipate the electromagnetic energy that it receives into the patient's body via the ring electrode 70. The shield wire 60 receives at least a portion of the radio-frequency energy that the lead 16 would otherwise be subject to, and therefore, reduces the quantity of radio-frequency energy received by the electrode wire 42. Using the shielding technique provided by embodiments of the present invention to reduce the radio frequency energy transmitted to the electrode wire 42, the quantity and/or amplitude of induced current that passes through the electrode 46 into the heart tissue maybe reduced. This may reduce the risk of heart tissue damage due to heating of the localized contact area between the electrode 46 and the heart.

It may be noted that in the embodiment of FIG. 9, shield wire 60 is not electrically coupled to case 26. In an alternative embodiment, shield wire may be coupled to one or more ring electrode 70 as well as to case 26.

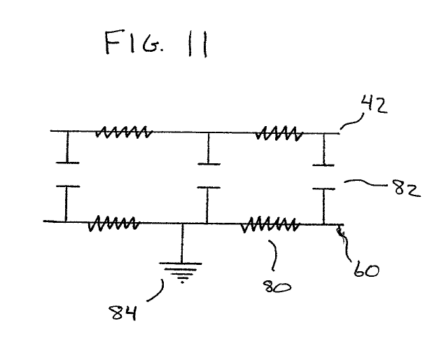

FIG. 10 shows a cross-sectional view of a portion of the embodiment of the invention shown in FIG. 10. More specifically, a cross-section of the lead 16 comprising a ring electrode 70 and a shield wire 62 is illustrated in FIG. 11. The unipolar lead 16 has a single electrode wire 42 and a shield wire 60 surrounded by an insulating material 44. The electrode wire 42 and the shield wire 60 generally run parallel within the lead 16, thereby providing shielding for the electrode wire 42. The shield wire 60 is electrically isolated from the electrode wire 42 by the insulating material 44 and is electrically connected to the ring electrode 70 by a connecting wire 72. The shield wire 60 is adapted to receive at least a portion of the radio-frequency energy that the lead 16 is subject to and therefore reduces the quantity and/or amplitude of radio-frequency energy that is received by the electrode wire 42. A substantial portion of the current that results in the shield wire 60 from the radio-frequency waves is transmitted through the connecting wire 72 into the ring electrode 70 where it is dissipated into the patient's body. The ring electrode 70 provides sufficient surface area for the current to pass into the body without creating a problem of excessive heating of the heart tissue. The particular embodiment shown in FIG. 11 is of a unipolar lead 16, but alternate embodiments of the present invention may have multiple electrode wires 42.

FIG. 11 is an electrical model of a lead 16 of an embodiment of the present invention. The electrode wire 42 and the shield wire 60 each have an impedance 80, which is the natural resistance to electrical transmission. The impedance 80 is primarily related to the material from which the wires are made. The two wires 42, 60 also have a capacitance 82 between them, which is a measure of the potential for electrical transmission to pass between the two. The capacitance 82 may be related to the physical distance between the wires and the type of material that separates them. The shield wire 60 is grounded 84 by a direct contact with the patient's body. As the lead 16 is exposed to electromagnetic waves, the shield wire 60 will act as a parasitic antenna and receive at least a portion of the electromagnetic energy that the electrode wire 42 would have received if the shield wire 60 were not present. The shield wire 60 receives at least a portion of the electromagnetic energy and transmits the energy through its ground 84 connection. The lead 16 is designed so that the two wires 42, 60 are spaced sufficiently apart and have an insulating material between each other such that the energy that is received within the shield wire 42 is not transmitted or “shorted” between the two. Using the electrical model described above (or other equivalent electrical circuits), current flow induced by electromagnetic waves is reduced, thereby reducing the possibility of damage in a patients body resulting from heating of the wires.

The particular embodiments disclosed above are illustrative only, as the invention may be modified and practiced in different but equivalent manners apparent to those skilled in the art having the benefit of the teachings herein. For example, in one embodiment, more than one shield wire may be provided. Furthermore, no limitations are intended to the details of construction or design herein shown, other than as described in the claims below. It is therefore evident that the particular embodiments disclosed above may be altered or modified and all such variations are considered within the scope of the invention. Accordingly, the protection sought herein is as set forth in the claims below.

What is claimed:

1. A medical electrical lead, comprising:
an electrode
an electrode conductor coupled to the electrode; and
a shield conductor adjacent to at least a portion of the electrode conductor, the shield conductor being electrically isolated from the electrode conductor and adapted to at least partially electrically shield the electrode wire.

2. The lead of claim 1, and further including at least one conductor to electrically coupled the shield conductor to a body.

3. The lead of claim 2, wherein the lead includes means to couple to an implantable medical device (IMD), and wherein the at least one conductor includes at least one surface of the IMD.

4. The lead of claim 2, wherein the at least one conductor includes at least one electrode.

5. The lead of claim 1, wherein the shield conductor comprises a biocompatible material.

6. The lead of claim 5, wherein the shield conductor comprises titanium.

7. The lead of claim 1, wherein the electrode conductor and shield conductor are coils.

8. The lead of claim 7, wherein the electrode conductor and shield conductor are arranged in a substantially co-axial manner.

9. The lead of claim 1, wherein the shield wire is selected to maintain the electrical lead stiffness.

10. An apparatus, comprising:
an implantable medical device;
an electrical lead electrically coupled to the implantable medical device; and
a shield wire disposed within the lead, the shield wire adapted to at least partially electrically shield the electrical lead.

11. The apparatus of claim 10, wherein the electrical lead comprises at least one electrical conductive electrode wire.

12. The apparatus of claim 11, wherein a layer of insulative material electrically insulates the shield wire and the electrode wire.

13. The apparatus of claim 10, and further including at least one conductor proximate a surface of the electrical lead, the at least one conductor being electrically coupled to the shield wire.

14. The apparatus of claim 13, wherein the at least one conductor is at least one ring electrode.

15. A method of shielding a medical electrical lead, comprising:
receiving electromagnetic energy within a shield wire carried by the lead; and
dissipating the energy into a patients body via at least one conductive surface electrically coupled to the shield wire.

16. A method of shielding a medical electrical lead from radio-frequency energy, comprising:
providing an electrical lead comprising at least one electrode wire and at least one shield wire; and
electrically coupling the at least one shield wire to a living body.

17. The method of claim 16, further comprising:
inserting the lead into the living body;
receiving radio-frequency energy within the at least one shield wire; and
dissipating the energy from the at least one shield wire into the living body.

18. A method of shielding a patient from the effects of electromagnetic waves on a medical electrical lead, comprising:
providing a lead having at least one electrode wire surrounded by a layer of insulative material;
providing a shield wire adjacent to, and electrically insulated from, the at least one electrode wire;
inserting the medical electrical lead within the body of the patient;
receiving electromagnetic waves with the shield wire; and
dissipating the energy received by the shield wire into the body to reduce current induced within the at least one electrode wire.

19. A method of manufacturing a shielded medical lead, comprising:
providing a lead having at least one electrode wire and at least one shield wire, wherein the at least one shield wire is adapted to receive electromagnetic waves; and
providing insulative material to carry the at least one electrode wire and the at least one shield wire such that the shield wire is not in direct electrical communication with the electrode wire.

20. The method of claim 19, further comprising:
electrically coupling the at least one shield wire to at least one conductive surface adapted to be in contact with the body.

21. The method of claim 20, further comprising:
electrically coupling the at least one shield wire to an electrode.

22. The method of claim 19, wherein the shield wire comprises a biocompatible material.
